(19) United States
(12) Patent Application Publication
(10) Pub. No.: US 2012/0029435 A1
(43) Pub. Date: Feb. 2, 2012

(54) NG/OG SUPPORT SYSTEMS

(76) Inventor: Miriam Gutierrez Del Rio, Fishers, IN (US)

(21) Appl. No.: 13/190,321

(22) Filed: Jul. 25, 2011

Related U.S. Application Data
(60) Provisional application No. 61/369,252, filed on Jul. 30, 2010.

(51) Int. Cl. A61M 39/00

(52) U.S. Cl. 604/179

(57) ABSTRACT

An external anchoring device for a nasogastric or orogastric tube comprising a cross shaped facial attachment base comprising a middle section, a top section, a bottom section, a left section and a right section; a first elongated fastening strap; and a second elongated fastening strap which are attached to and extend downwardly from the bottom section of the cross shaped facial attachment base; a first fastener, and a second fastener. The cross shaped facial attachment base, the first elongated fastening strap, and the second elongated fastening strap form a unitary piece which comprises an outside layer, an adhesive layer and a covering layer. The cross shaped facial attachment base is affixed to a user's nose and the first elongated fastening strap and the second elongated fastening strap may be wrapped around and adhered to a nasogastric tube and secured to each other via the first fastener and the second fastener.

### CROSS-REFERENCE TO RELATED APPLICATION

The present application is related to and claims priority from prior provisional application Ser. No. 61/369,252, filed Jul. 31, 2010 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of nasogastric or orogastric tubes and more specifically relates to a nasogastric or orogastric tube holder.

2. Description of the Related Art

A nasogastric tube is used for feeding to place nutrients directly into the stomach when a patient cannot take food or drink by mouth, or for administering drugs and other oral agents. The nasogastric tube may further be used to remove the contents of the stomach, including air, to decompress the stomach, or to remove small solid objects and fluid, such as poison, from the stomach. The nasogastric tube is inserted through a patient's nose, is passed down the esophagus, and into the stomach. The orogastric tube is the same as a nasogastric tube, except that it is passed through the mouth rather than the nose.

Since a nasogastric or orogastric tube may move due to a patient's activity or motion of the stomach, coughing, chocking, etc., tape is typically used to secure the nasogastric or orogastric tube to the patient's face and to hold the nasogastric or orogastric tube in place. Unfortunately, this method for holding nasogastric or orogastric tubes in place may be ineffective. Tape may not provide optimal staying power. The tape may come loose, allowing the nasogastric or orogastric tube to be easily pulled out or displaced.

If a nasogastric or orogastric tube is pulled out or does not stay in the proper place this may cause nasal irritation or pressure necrosis and medical staff members are then required to re-tape the tubes in place. The process of repeatedly removing and applying tape to the face may cause discomfort to the patient. In cases of severe displacement, the tubes can become dislodged, requiring medical staff to reinsert the tubes which may cause great discomfort and trauma to patients, and which is time consuming for medical personnel.

Therefore a need exist for a nasogastric or orogastric tube holder which keeps a nasogastric or orogastric tube in the proper position, causes no discomfort to the patient, and is easy to use.

Various attempts have been made to solve the above-mentioned problems such as those found in U.S. Pat. Nos. 4,282,871, 4,480,639, 5,117,818, 3,972,321, 4,932,943, and 5,156,641. This prior art is representative of nasogastric or orogastric tube holders. None of the above inventions and patents, taken either singly or in combination, is seen to describe the invention as claimed.

Ideally, a nasogastric or orogastric tube holder should require no maintenance and, yet, would operate reliably and be manufactured at a modest expense. Thus, a need exists for a reliable NG/OG Support System to hold a nasogastric or orogastric tube in the proper position and to avoid the above-mentioned problems.

### BRIEF SUMMARY OF THE INVENTION

In view of the foregoing disadvantages inherent in the known nasogastric or orogastric tube holder art, the present invention provides a novel NG/OG Support System. The general purpose of the present invention, which will be described subsequently in greater detail, is to provide a NG/OG Support System to reliably hold a nasogastric or orogastric tube in the proper position.

Disclosed is an external anchoring device for a nasogastric or orogastric tube comprising a cross shaped facial attachment base comprising a middle section, a top section, a bottom section, a left section and a right section; a first elongated fastening strap; a second elongated fastening strap; a first fastener and a second fastener.

The cross shaped facial attachment base, the first elongated fastening strap, and the second elongated fastening strap comprise a unitary piece and the first elongated fastening strap and the second elongated fastening strap are attached to and extend downwardly from the bottom section of the cross shaped facial attachment base. The cross shaped facial attachment base, the first elongated fastening strap, and the second elongated fastening strap comprise an outside layer, an adhesive layer and a covering layer wherein the outside layer, the adhesive layer and the covering layer comprise a one piece construction. The first fastener is attached to the end of the first elongated fastening strap and the second fastener is attached to the end of the second elongated fastening strap.

The cross shaped facial attachment base may be affixed to a user's nose and the first elongated fastening strap and the second elongated fastening strap may be wrapped around and adhered to a nasogastric tube and secured to each other via the first fastener and the second fastener so as to hold the nasogastric tube in place on a face of the user and thereby preventing the nasogastric tube from being displaced. Alternatively, the cross shaped facial attachment base may be affixed to an endotracheal tube and an orogastric tube so as to secure them together as one unit and the first elongated fastening strap and the second elongated fastening strap may be wrapped around and adhered to the endotracheal tube and the orogastric tube and the first elongated fastening strap and the second elongated fastening strap may be secured to each other via the first fastener and the second fastener.

A preferred method of use is further disclosed herein comprising the following steps: positioning a nasogastric tube into a user's nose; peeling a covering layer off an adhesive layer of the external anchoring device for a nasogastric or orogastric tube; adhesively adhering a cross shaped facial attachment base of the external anchoring device for a nasogastric or orogastric tube to the user's nose; adhesively adhering a first elongated fastening strap and a second elongated fastening strap of the external anchoring device for a nasogastric or orogastric tube to the nasogastric tube; fastening the first elongated fastening strap and the second elongated fastening strap to each other via the first fastener and the second fastener; disconnecting the first fastener and the second fastener; removing the external anchoring device for a nasogastric or orogastric tube from a nasogastric tube; and removing the nasogastric tube from the user's mouth or nose.

The present invention holds significant improvements and serves as a NG/OG Support System. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, NG/OG Support Systems, constructed and operative according to the teachings of the present invention.

FIG. 1 shows a perspective view illustrating an external anchoring device for a nasogastric or orogastric tube according to an embodiment of the present invention.

FIG. 2 is a perspective view illustrating the external anchoring device for a nasogastric or orogastric tube according to an embodiment of the present invention of FIG. 1.

FIG. 3 is a perspective view illustrating the external anchoring device for a nasogastric or orogastric tube in an in-use condition according to an embodiment of the present invention of FIG. 1.

FIG. 4 is a perspective view illustrating the external anchoring device for a nasogastric or orogastric tube in an in-use condition according to an embodiment of the present invention of FIG. 1.

FIG. 5 is a perspective view illustrating the external anchoring device for a nasogastric or orogastric tube according to an embodiment of the present invention of FIG. 1.

FIG. 6 is a flowchart illustrating a method of using an external anchoring device for a nasogastric or orogastric tube according to an embodiment of the present invention of FIG. 1.

The various embodiments of the present invention will hereinafter be described in conjunction with the appended drawings, wherein like designations denote like elements.

### DETAILED DESCRIPTION

As discussed above, embodiments of the present invention relate to nasogastric or orogastric tubes and more particularly to a nasogastric or orogastric tube holder as used to hold a nasogastric or orogastric tube in the proper position and to avoid movements of a nasogastric or orogastric tube.

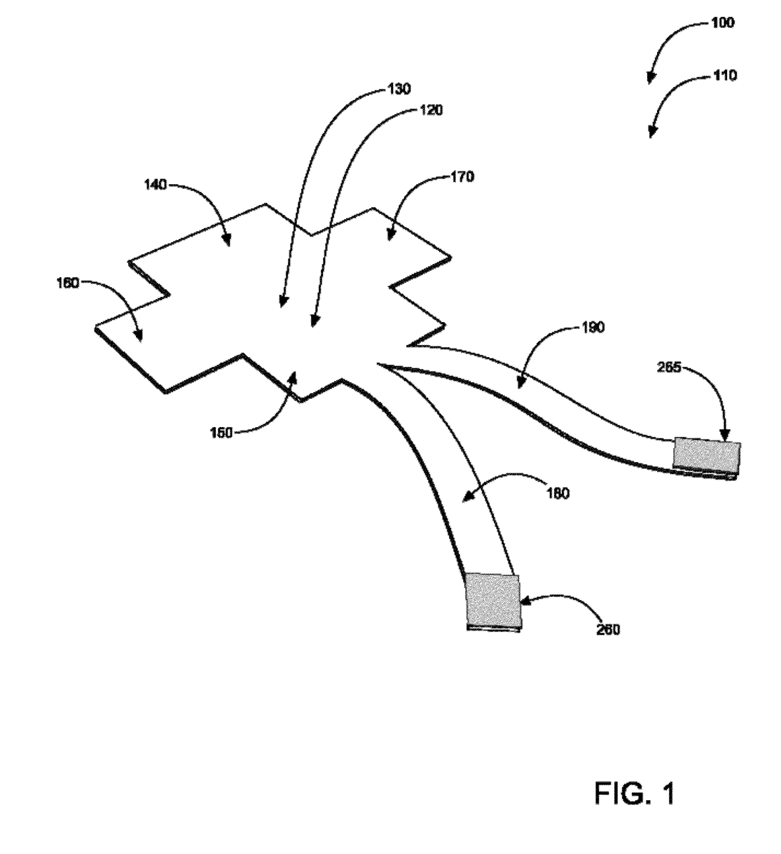

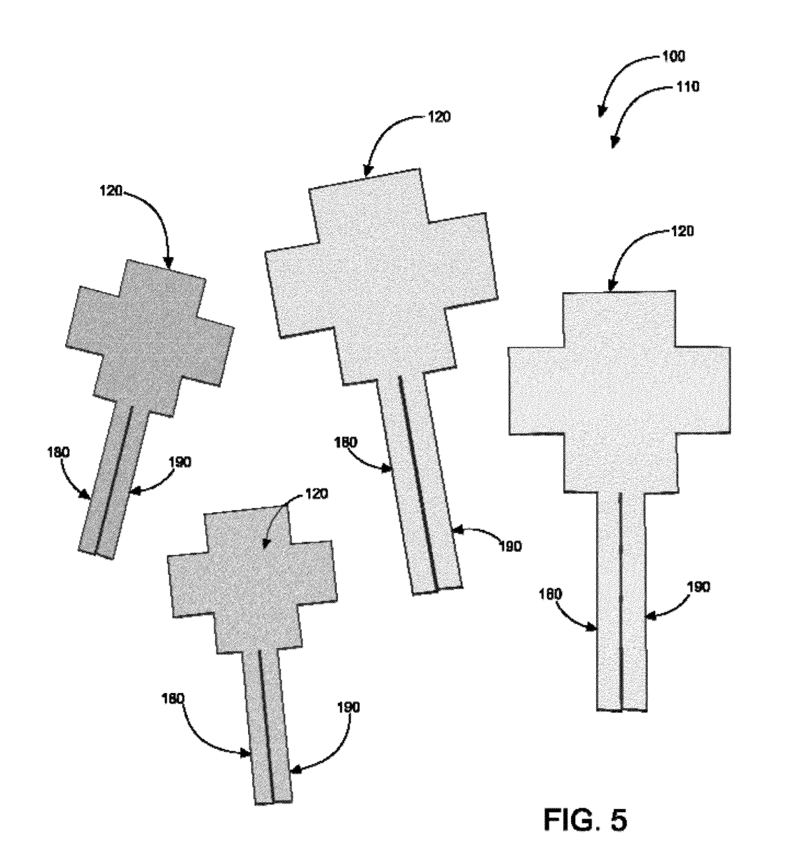

Referring to the drawings by numerals of reference there is shown in FIG. 1 and FIG. 5, perspective views illustrating an external anchoring device for a nasogastric or orogastric tube 110 according to an embodiment of the present invention.

External anchoring device for a nasogastric or orogastric tube 110 preferably comprises cross shaped facial attachment base 120 comprising middle section 130, top section 140, bottom section 150, left section 160 and right section 170; first elongated fastening strap 180; second elongated fastening strap 190; and first fastener 260 and second fastener 265.

Cross shaped facial attachment base 120 comprises middle section 130, top section 140, bottom section 150, left section 160 and right section 170. Middle section 130, top section 140 and bottom section 150 form an elongated main component of external anchoring device for a nasogastric or orogastric tube 110 which may rest on the bridge of user's 230 nose. Left section 160 and right section 170 comprise side elements which provide additional surfaces to attach external anchoring device for a nasogastric or orogastric tube 110 to the two sides of user's 230 nose. Cross shaped facial attachment base 120 preferably comprises the shape of a cross with straight edges. However, cross shaped facial attachment base 120 may alternatively comprise rounded edges. Cross shaped facial attachment base 120 preferably comprises a size suitably matched to the nose of user 230 wherein user 230 is a child or an adult.

First elongated fastening strap 180 and second elongated fastening strap 190 are attached to and extend downwardly from bottom section 150 of cross shaped facial attachment base 120 and preferably comprise two narrow elongated straps. Cross shaped facial attachment base 120, first elongated fastening strap 180, and second elongated fastening strap 190 thereby comprise a unitary piece. First elongated fastening strap 180 and second elongated fastening strap 190 preferably extend downwardly from bottom section 150 of cross shaped facial attachment base 120 in parallel adjacent alignment. Alternatively, first elongated fastening strap 180 and second elongated fastening strap 190 may extend downwardly from bottom section 150 of cross shaped facial attachment base 120 in an angle with respect to each other.

First elongated fastening strap 180 and second elongated fastening strap 190 may comprise first fastener 260 and second fastener 265. First fastener 260 is situated on the adhesive layer 210 at the end of first elongated fastening strap 180 and second fastener 265 is situated on the adhesive layer 210 at the end of second elongated fastening strap 190. First fastener 260 and second fastener 265 preferably comprise two complementary fasteners which may be attached to each other. First fastener 260 and second fastener 265 preferably comprise Velcro. However, first fastener 260 and second fastener 265 may also comprise mechanical fasteners or snap fasteners.

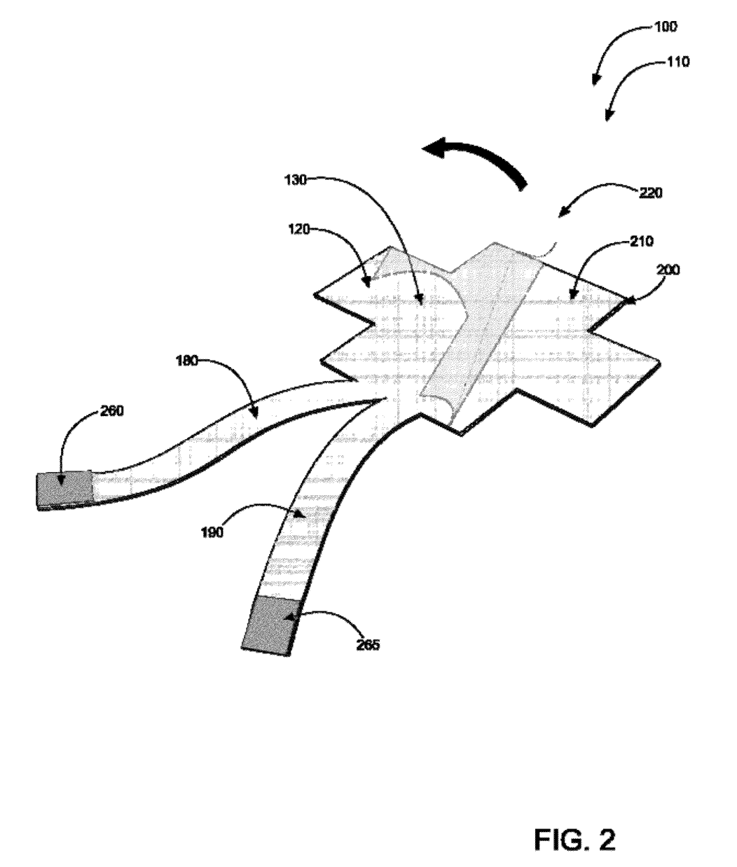

Referring now to FIG. 2, a perspective view illustrating the external anchoring device for a nasogastric or orogastric tube 110 according to an embodiment of the present invention of FIG. 1.

Cross shaped facial attachment base 120, first elongated fastening strap 180, and second elongated fastening strap 190 each comprise an outside layer 200, an adhesive layer 210, and a covering layer 220. Outside layer 200, adhesive layer 210, and covering layer 220 preferably comprise a one piece construction.

Outside layer 200 preferably comprises a thin, flexible, and deformable elastic sheet. Outside layer 200 preferably is made of sheer vinyl. Alternatively, outside layer 200 may comprise fabric, such as cotton. Outside layer 200 preferably is beige for the adult-size external anchoring device for a nasogastric or orogastric tube 110 and pink or blue for the child-size external anchoring device for a nasogastric or orogastric tube 110.

Cross shaped facial attachment base 120, first elongated fastening strap 180, and second elongated fastening strap 190 comprise adhesive layer 210 so as to facilitate the attachment of cross shaped facial attachment base 120 to nose of user 230 or to orogastric tube 250 and endotracheal tube 270 and the attachment of first elongated fastening strap 180 and second elongated fastening strap to nasogastric tube 240 or orogastric tube 250 and endotracheal tube 270. Adhesive layer 210 preferably comprises a skin friendly and suitable adhesive material. Adhesive layer 210 is removably covered by covering layer 220 which may be peeled away from adhesive layer 210 thereby exposing a contact-adhesive surface suitable to adhere to a skin surface on a face of user 230.

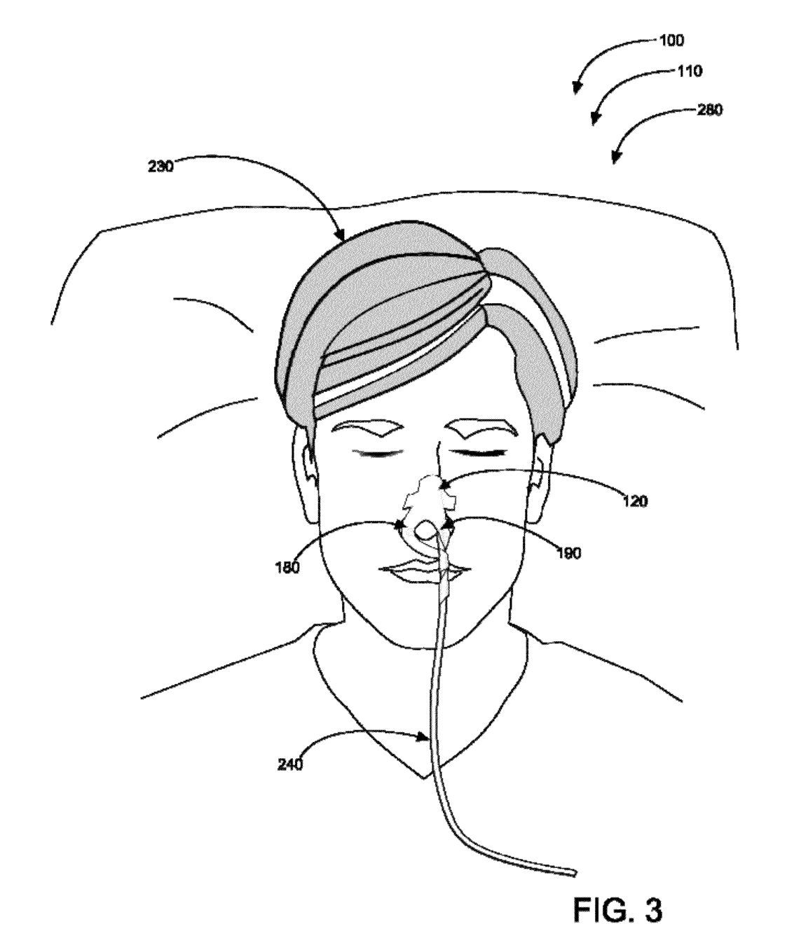

Referring now to FIG. 3, a perspective view illustrating the external anchoring device for a nasogastric or orogastric tube 110 in an in-use condition 280 according to an embodiment of the present invention of FIG. 1.

Covering layer 220 may be peeled away from adhesive layer 210 thereby exposing a contact-adhesive surface suitable to adhere to a skin surface on the face of user 230. Cross shaped facial attachment base 120 may be affixed to user's 230 nose and first elongated fastening strap 180 and second elongated fastening strap 190 may be wrapped around and adhered to nasogastric tube 240 and secured to each other via first fastener 260 and second fastener 265 so as to hold nasogastric tube 240 in place on face of user 230 and thereby preventing nasogastric tube 240 from being displaced. First fastener 260 and second fastener 265 comprise matching Velcro so as facilitate easy removal or readjustment of first elongated fastening strap 180 and second elongated fastening strap 190 and external anchoring device for a nasogastric or orogastric tube 110.

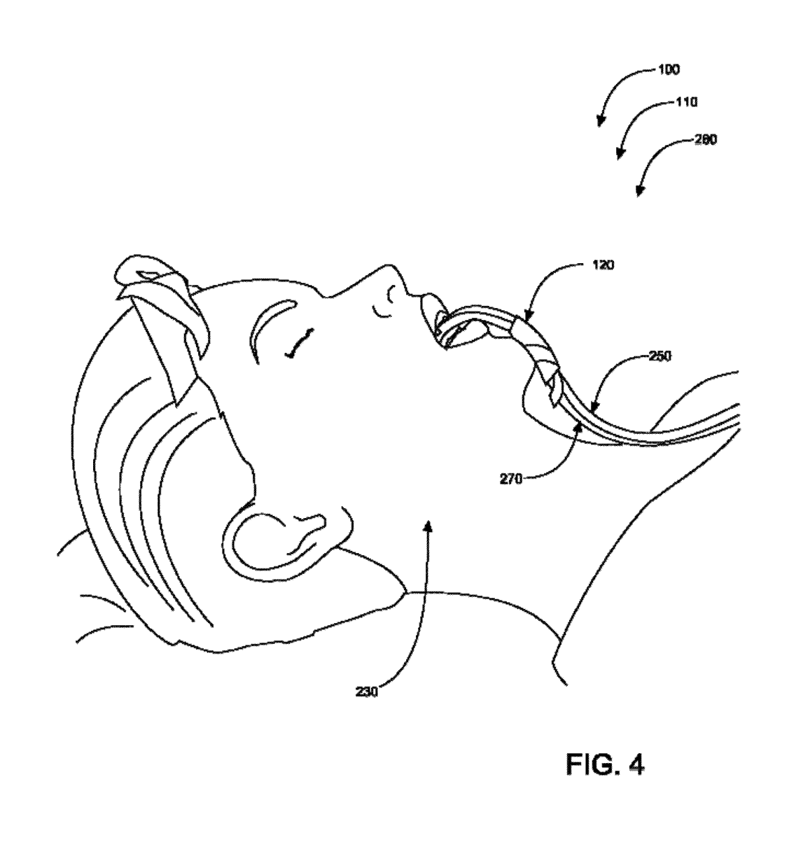

Referring now to FIG. 4, a perspective view illustrating the external anchoring device for a nasogastric or orogastric tube 110 in an in-use condition 280 according to an embodiment of the present invention of FIG. 1.

Covering layer 220 may be peeled away from adhesive layer 210 thereby exposing a contact-adhesive surface suitable to adhere to a skin surface on the face of user 230. Cross shaped facial attachment base 120 may then be affixed to an endotracheal tube 270 and an orogastric tube 250 so as to secure them together as one unit and first elongated fastening strap 180 and second elongated fastening strap 190 may be wrapped around and adhered to endotracheal tube 270 and orogastric tube 250 and first elongated fastening strap 180 and second elongated fastening strap 190 may then be secured to each other via first fastener 260 and second fastener 265.

NG/OG Support Systems 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other contents or arrangements such as, for example, including more or less components, customized parts, different color combinations, parts may be sold separately, etc., may be sufficient.

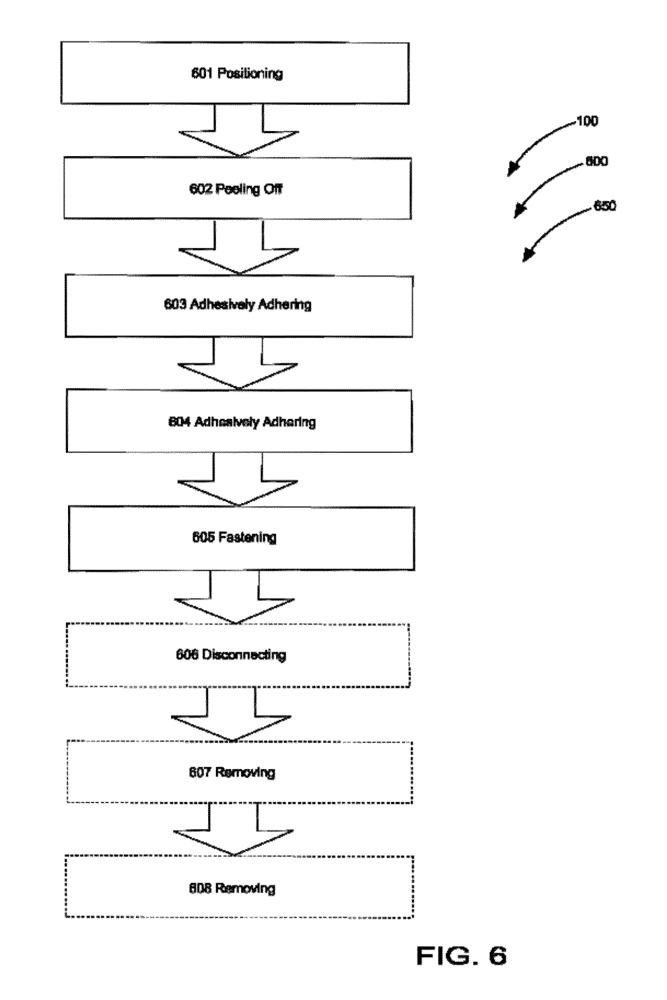

Referring now to FIG. 6, a flowchart 650 illustrating a method 600 of using an external anchoring device for a nasogastric or orogastric tube 110 according to an embodiment of the present invention of FIG. 1.

In accordance with the embodiments of the present invention a preferred method of use 600 is disclosed herein comprising the following steps: step one 601 positioning a nasogastric tube 240 into a user's 230 nose; step two 602 peeling covering layer 220 off adhesive layer 210 of external anchoring device for a nasogastric or orogastric tube 110; step three 603 adhesively adhering cross shaped facial attachment base 120 of external anchoring device for a nasogastric or orogastric tube 110 to user's 230 nose; step four 604 adhesively adhering first elongated fastening strap 180 and second elongated fastening strap 190 of external anchoring device for a nasogastric or orogastric tube 110 to nasogastric tube 240; step five 605 fastening first elongated fastening strap 180 and second elongated fastening strap 190 to each other via first fastener 260 and second fastener 265; step six 606 disconnecting first fastener 260 and second fastener 265; step seven 607 removing external anchoring device for a nasogastric or orogastric tube 110 from nasogastric tube 240; and step eight 608 removing nasogastric tube 240 from user's 230 nose.

It should be noted that steps six 606, seven 607, and eight 608 are optional steps and may not be implemented in all cases. Optional steps of method 600 are illustrated using dotted lines in FIG. 6 so as to distinguish them from the other steps of method 600.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An external anchoring device for a nasogastric or orogastric tube comprising:
a cross shaped facial attachment base comprising a middle section, a top section, a bottom section, a left section and a right section;
a first elongated fastening strap;
a second elongated fastening strap; and
a first fastener and a second fastener;
wherein said cross shaped facial attachment base, said first elongated fastening strap, and said second elongated fastening strap comprise a unitary piece;
wherein said first elongated fastening strap and said second elongated fastening strap are attached to and extend downwardly from said bottom section of said cross shaped facial attachment base;
wherein said cross shaped facial attachment base, said first elongated fastening strap, and said second elongated fastening strap each comprise an outside layer, an adhesive layer and a covering layer wherein said outside layer, said adhesive layer and said covering layer comprise a one piece construction;
wherein said first fastener is attached to the end of said first elongated fastening strap and said second fastener is attached to the end of said second elongated fastening strap; and
wherein said cross shaped facial attachment base may be affixed to a user's nose and said first elongated fastening strap and said second elongated fastening strap may be wrapped around and adhered to a nasogastric tube and secured to each other via said first fastener and said second fastener so as to hold said nasogastric tube in place on a face of said user and thereby preventing said nasogastric tube from being displaced.

2. The external anchoring device for a nasogastric or orogastric tube of claim 1 wherein said cross shaped facial attachment base may be affixed to an endotracheal tube and an orogastric tube so as to secure them together as one unit and said first elongated fastening strap and said second elongated fastening strap may be wrapped around and adhered to said endotracheal tube and said orogastric tube and said first elongated fastening strap and said second elongated fastening strap may be secured to each other via said first fastener and said second fastener.

3. The external anchoring device for a nasogastric or orogastric tube of claim 1 wherein said first fastener and said second fastener comprise Velcro.

4. The external anchoring device for a nasogastric or orogastric tube of claim 1 wherein said outside layer of said cross shaped facial attachment base and said outside layer of said first elongated fastening strap and said outside layer of said second elongated fastening strap comprise a thin, flexible, and deformable elastic sheet.

5. The external anchoring device for a nasogastric or orogastric tube of claim 4 wherein said thin, flexible, and deformable elastic sheet comprises vinyl.

6. The external anchoring device for a nasogastric or orogastric tube of claim 4 wherein said thin, flexible, and deformable elastic sheet comprises plastic.

7. The external anchoring device for a nasogastric or orogastric tube of claim 4 wherein said thin, flexible, and deformable elastic sheet comprises cotton.

8. The external anchoring device for a nasogastric or orogastric tube of claim 4 wherein said thin, flexible, and deformable elastic sheet is beige.

9. The external anchoring device for a nasogastric or orogastric tube of claim 4 wherein said thin, flexible, and deformable elastic sheet is pink.

10. The external anchoring device for a nasogastric or orogastric tube of claim 4 wherein said thin, flexible, and deformable elastic sheet is blue.

11. The external anchoring device for a nasogastric or orogastric tube of claim 1 wherein said adhesive layer is removably covered by said covering layer and said covering layer may be peeled away from said adhesive layer thereby exposing a contact-adhesive surface suitable to adhere to a skin surface on said face of said user.

12. The external anchoring device for a nasogastric or orogastric tube of claim 1 wherein said cross shaped facial attachment base comprises rounded edges.

13. The external anchoring device for a nasogastric or orogastric tube of claim 1 wherein said first elongated fastening strap and said second elongated fastening strap extend downwardly from said bottom section of said cross shaped facial attachment base in parallel adjacent alignment.

14. The external anchoring device for a nasogastric or orogastric tube of claim 1 wherein said first elongated fastening strap and said second elongated fastening strap extend downwardly from said bottom section of said cross shaped facial attachment base in an angle with respect to each other.

15. The external anchoring device for a nasogastric or orogastric tube of claim 1 wherein said anchoring device for a nasogastric or orogastric tube is repositionable.

16. The external anchoring device for a nasogastric or orogastric tube of claim 1 wherein said anchoring device for a nasogastric or orogastric tube comprises a size suitably matched to said face of said user wherein said user is a child.

17. The external anchoring device for a nasogastric or orogastric tube of claim 1 wherein said anchoring device for a nasogastric or orogastric tube comprises a size suitably matched to said face of said user wherein said user is an adult.

18. An external anchoring device for a nasogastric or orogastric tube comprising:
a cross shaped facial attachment base comprising a middle section, a top section, a bottom section, a left section and a right section;
a first elongated fastening strap;
a second elongated fastening strap; and
a first Velcro fastener and a second Velcro fastener;
wherein said cross shaped facial attachment base, said first elongated fastening strap, and said second elongated fastening strap comprise a unitary piece;
wherein said first elongated fastening strap and said second elongated fastening strap are attached to and extend downwardly from said bottom section of said cross shaped facial attachment base in parallel adjacent alignment;
wherein said cross shaped facial attachment base, said first elongated fastening strap, and said second elongated fastening strap each comprise an outside layer, an adhesive layer and a covering layer wherein said outside layer, said adhesive layer and said covering layer comprise a one piece construction and said outside layer comprises a thin, flexible, and deformable elastic sheet comprising beige vinyl;
wherein said adhesive layer is removably covered by said covering layer and said covering layer may be peeled away from said adhesive layer thereby exposing a contact-adhesive surface suitable to adhere to a skin surface on said face of said user;
wherein said first Velcro fastener is attached to the end of said first elongated fastening strap and said second Velcro fastener is attached to the end of said second elongated fastening strap; and
wherein said cross shaped facial attachment base may be affixed to a user's nose and said first elongated fastening strap and said second elongated fastening strap may be wrapped around and adhered to a nasogastric tube and secured to each other via said first fastener and said second fastener so as to hold said nasogastric tube in place on a face of said user and thereby preventing said nasogastric tube from being displaced.

19. A method of using an external anchoring device for a nasogastric or orogastric tube comprising the steps of:
positioning a nasogastric tube into a user's nose;
peeling a covering layer off an adhesive layer of said external anchoring device for a nasogastric or orogastric tube;
adhesively adhering a cross shaped facial attachment base of said external anchoring device for a nasogastric or orogastric tube to said user's nose;
adhesively adhering a first elongated fastening strap and a second elongated fastening strap of said external anchoring device for a nasogastric or orogastric tube to said nasogastric tube;
fastening said first elongated fastening strap and said second elongated fastening strap to each other via said first fastener and said second fastener;
disconnecting said first fastener and said second fastener;
removing said external anchoring device for a nasogastric or orogastric tube from a nasogastric tube; and
removing said nasogastric tube from said user's nose.
